(19) United States
(12) Patent Application Publication
(10) Pub. No.: US 2024/0407765 A1
(43) Pub. Date: Dec. 12, 2024

(54) ULTRASOUND DIAGNOSIS APPARATUS

(71) Applicant: CANON MEDICAL SYSTEMS CORPORATION, Otawara-shi (JP)

(72) Inventors: Hiroki TAKAHASHI, Nasushiobara (JP); Takeshi FUKASAWA, Nasushiobara (JP); Akihiro KAKEE, Nasushiobara (JP)

(73) Assignee: CANON MEDICAL SYSTEMS CORPORATION, Otawara-shi (JP)

(21) Appl. No.: 18/809,967

(22) Filed: Aug. 20, 2024

Related U.S. Application Data
(62) Division of application No. 17/214,991, filed on Mar. 29, 2021.

(30) Foreign Application Priority Data
Mar. 31, 2020 (JP) 2020-063422

(51) Int. Cl. A61B 8/08; A61B 8/00; A61B 8/06

(52) U.S. Cl. CPC A61B 8/5246; A61B 8/06; A61B 8/461

(57) ABSTRACT

According to one embodiment, an ultrasound diagnosis apparatus includes processing circuitry. The processing circuitry acquires a change in a display parameter related to image display in a first bloodstream imaging mode of displaying a bloodstream signal acquired by an ultrasonic probe during execution of the first bloodstream imaging mode, and determines whether or not the display parameter after the change is more suitable for a second bloodstream imaging mode than for the first bloodstream imaging mode, an imaging method of the second bloodstream imaging mode differing from an imaging method of the first bloodstream imaging mode.

### CROSS-REFERENCE TO RELATED APPLICATIONS

This application is a divisional Application of U.S. Application Ser. No. 17/214,991, filed Mar. 29, 2021, which is based upon and claims the benefit of priority from Japanese Patent Application No. 2020-063422, filed Mar. 31, 2020, the entire contents of which are incorporated herein by reference.

### FIELD

Embodiments described herein relate generally to an ultrasound diagnosis apparatus.

### BACKGROUND

Traditionally, ultrasound diagnosis apparatuses are sometimes equipped with multiple bloodstream imaging modes for displaying a bloodstream using a two-dimensional image. Those bloodstream imaging modes include, for example, a color Doppler imaging (CDI) mode that allows for display of flow rate information of a bloodstream and a power Doppler mode that allows for display of power information of a bloodstream. The bloodstream imaging modes also include various modes such as a mode specialized for representing a low flow rate and a mode which allows for display with high sensitivity. A user needs to use the different bloodstream imaging modes in accordance with the clinical information that the user wishes to observe.

However, when the user uses the different bloodstream imaging modes in accordance with the clinical information that the user wishes to observe, the user needs to know the respective bloodstream imaging modes for obtaining desired clinical information. This not only imposes a burden on the user but may also prevent the user from selecting an appropriate bloodstream imaging mode.

### BRIEF DESCRIPTION OF THE DRAWINGS

FIG. 1 is a block diagram showing a configuration example of an ultrasound diagnosis apparatus according to a first embodiment.

FIG. 2 is a perspective view of an exterior of an apparatus main body of the ultrasound diagnosis apparatus in the first embodiment.

FIG. 3 is a top view of an exterior of an input device connected to the ultrasound diagnosis apparatus in the first embodiment.

FIG. 4 is a flowchart for explaining operations of processing circuitry that executes bloodstream imaging mode transition processing in the first embodiment.

FIG. 5 is a diagram illustrating a display image including a display example for giving notice of a transition to a recommended bloodstream imaging mode in the first embodiment.

FIG. 6 is a diagram illustrating a display image including a display example indicating that a transition to a recommended bloodstream imaging mode has been made in the first embodiment.

FIG. 7 is a diagram for explaining the bloodstream imaging mode transition processing in the first embodiment.

FIG. 8 is a diagram for describing a first specific example of the bloodstream imaging mode transition processing in the first embodiment.

FIG. 9 is a diagram for describing a second specific example of the bloodstream imaging mode transition processing in the first embodiment.

FIG. 10 is a diagram for describing a third specific example of the bloodstream imaging mode transition processing in the first embodiment.

FIG. 11 is a flowchart for explaining operations of processing circuitry that executes bloodstream imaging mode transition processing in an application example of the first embodiment.

FIG. 12 is a diagram illustrating a display image including a display example for prompting a transition to a recommended bloodstream imaging mode in the application example of the first embodiment.

FIG. 13 is a diagram illustrating a display image of a touch panel including a software button for transitioning to a recommended bloodstream imaging mode in the application example of the first embodiment.

FIG. 14 is a diagram illustrating a display image including options and a display example for prompting a transition to a recommended bloodstream imaging mode in the application example of the first embodiment.

FIG. 15 is a flowchart for explaining operations of processing circuitry that executes bloodstream imaging method transition processing in a second embodiment.

FIG. 16 is a diagram for explaining the bloodstream imaging method transition processing in the second embodiment.

FIG. 17 is a diagram for describing a first specific example of the bloodstream imaging method transition processing in the second embodiment.

FIG. 18 is a diagram for describing a second specific example of the bloodstream imaging method transition processing in the second embodiment.

FIG. 19 is a diagram for describing a third specific example of the bloodstream imaging method transition processing in the second embodiment.

### DETAILED DESCRIPTION

In general, according to one embodiment, an ultrasound diagnosis apparatus includes processing circuitry. The processing circuitry acquires a change in a display parameter related to image display in a first bloodstream imaging mode of displaying a bloodstream signal acquired by an ultrasonic probe during execution of the first bloodstream imaging mode, and determines whether or not the display parameter after the change is more suitable for a second bloodstream imaging mode than for the first bloodstream imaging mode, an imaging method of the second bloodstream imaging mode differing from an imaging method of the first bloodstream imaging mode.

Hereinafter, embodiments of an ultrasound diagnosis apparatus will be described in detail with reference to the accompanying drawings.

### First Embodiment

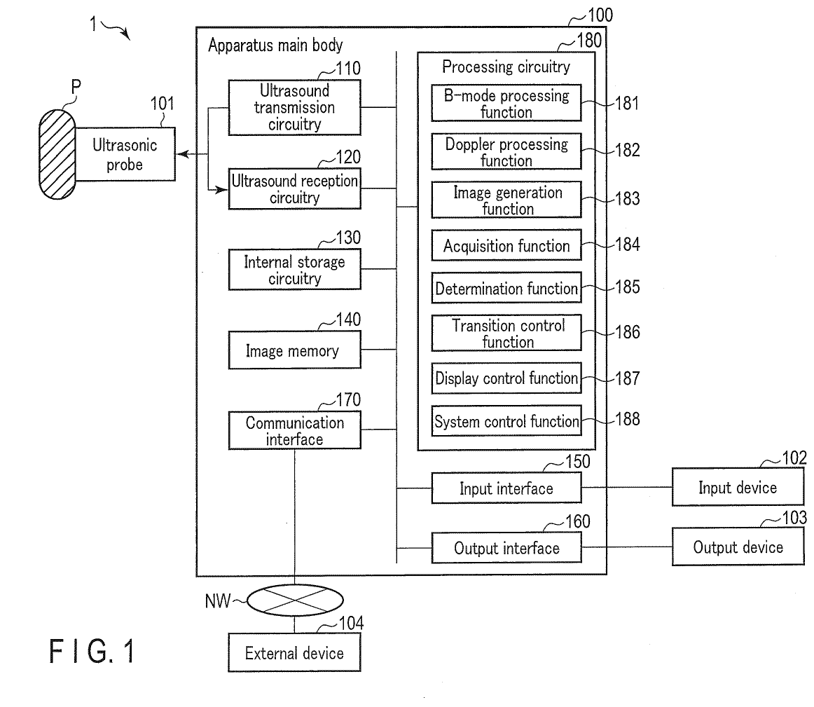

FIG. 1 is a block diagram showing a configuration example of an ultrasound diagnosis apparatus according to a first embodiment. An ultrasound diagnosis apparatus 1 shown in FIG. 1 includes an apparatus main body 100 and an ultrasonic probe 101. The apparatus main body 100 is connected to an input device 102 and an output device 103. The apparatus main body 100 is connected to an external device 104 via a network NW. The external device 104 is, for example, a server equipped with a picture archiving and communication system (PACS).

The ultrasonic probe 101 executes ultrasound scanning in a scan area of a living body P, which is a subject, under the control of, for example, the apparatus main body 100. The ultrasonic probe 101 includes, for example, a plurality of piezoelectric vibrators, a matching layer provided between a case and the plurality of piezoelectric vibrators, and a backing material that prevents ultrasound waves from propagating backward with respect to a radiation direction from the piezoelectric vibrators. The ultrasonic probe 101 is, for example, a one-dimensional array linear probe in which a plurality of ultrasound transducers are arranged in a predetermined direction. The ultrasonic probe 101 is detachably connected to the apparatus main body 100. The ultrasonic probe 101 may be provided with buttons which are pressed when offset processing, an operation for freezing an ultrasound image (freeze operation), and the like are performed.

The piezoelectric vibrators generate an ultrasound wave based on a drive signal supplied from ultrasound transmission circuitry 110 (described later) of the apparatus main body 100. An ultrasound wave is thereby transmitted from the ultrasonic probe 101 to the living body P. When an ultrasound wave is transmitted from the ultrasonic probe 101 to the living body P, the transmitted ultrasound wave is sequentially reflected by a discontinuous surface of the acoustic impedance of the body tissue of the living body P, and is received as a reflected wave signal by the plurality of piezoelectric vibrators. The amplitude of the received reflected wave signal depends on the difference in the acoustic impedance on the discontinuous surface from which the ultrasound wave is reflected. If the transmitted ultrasound pulse is reflected by a moving bloodstream or surface of a cardiac wall or the like, the frequency of the resultant reflected wave signal will be shifted, due to the Doppler effect, depending on the velocity component of the moving object in the ultrasonic transmission direction. The ultrasonic probe 101 receives the reflected wave signal from the living body P, and converts it into an electric signal.

FIG. 1 illustrates a connection relationship between a single ultrasonic probe 101 and the apparatus main body 100. However, the apparatus main body 100 can be connected to a plurality of ultrasonic probes. Which of the connected ultrasonic probes is to be used for the ultrasound scanning can be selected discretionarily by using, for example, a software button on a touch panel described later.

The apparatus main body 100 generates an ultrasound image based on the reflected wave signal received by the ultrasonic probe 101. The apparatus main body 100 includes ultrasound transmission circuitry 110, ultrasound reception circuitry 120, internal storage circuitry 130, an image memory 140, an input interface 150, an output interface 160, a communication interface 170, and processing circuitry 180.

The ultrasound transmission circuitry 110 is a processor that supplies a drive signal to the ultrasonic probe 101. The ultrasound transmission circuitry 110 is implemented by, for example, trigger generation circuitry, delay circuitry, and pulser circuitry. The trigger generation circuitry repeatedly generates a rate pulse for forming a transmission ultrasound wave at a predetermined rate frequency. The delay circuitry gives each rate pulse generated by the trigger generation circuitry a delay time for each piezoelectric vibrator needed to converge the ultrasound waves generated from the ultrasonic probe into a beam and determine the transmission directivity. The pulser circuitry applies a drive signal (drive pulse) to the multiple ultrasound transducers of the ultrasonic probe 101 at a timing based on the rate pulse. The transmission direction from the surfaces of the piezoelectric vibrators can be discretionarily adjusted by varying the delay time given to each rate pulse by the delay circuitry.

The ultrasound transmission circuitry 110 can discretionarily change the output intensity of the ultrasound wave through a drive signal. In the ultrasound diagnosis apparatus, the influence of the attenuation of the ultrasound wave in the living body P can be reduced by increasing the output intensity. By reducing the influence of the attenuation of the ultrasound wave, the ultrasound diagnosis apparatus can obtain a reflected wave signal having a large S/N ratio when receiving the signal.

In general, when the ultrasound wave is propagated inside the living body P, the strength of the oscillation of the ultrasound wave corresponding to the output intensity (the strength is also referred to as “acoustic power”) is attenuated. The attenuation of the acoustic power is caused by absorption, scattering, reflection, and the like. Also, the degree of reduction of the acoustic power depends on the frequency of the ultrasound wave and the distance of the ultrasound wave in the radiation direction. For example, the degree of attenuation is increased by increasing the frequency of the ultrasound wave. Also, the longer the distance of the ultrasound wave in the radiation direction, the larger the degree of attenuation.

The ultrasound reception circuitry 120 is a processor that performs various types of processing on the reflected wave signal received by the ultrasonic probe 101, and thereby generates a reception signal. The ultrasound reception circuitry 120 generates a reception signal for the reflected wave signal of the ultrasound wave obtained by the ultrasonic probe 101. Specifically, the ultrasound reception circuitry 120 is implemented by, for example, a preamplifier, an A/D converter, a demodulator, and a beam former. The preamplifier performs gain correction processing by amplifying the reflected wave signal received by the ultrasonic probe 101 for each channel. The A/D converter converts the gain-corrected reflected wave signal into a digital signal. The demodulator demodulates the digital signal. The beam former, for example, gives the demodulated digital signal a delay time needed to determine the reception directivity, and adds a plurality of digital signals given the delay time. Through the addition processing by the beam former, a reception signal with an enhanced reflection component in a direction corresponding to the reception directivity is generated.

The internal storage circuitry 130 includes, for example, a magnetic storage medium, an optical storage medium, or a processor-readable storage medium such as a semiconductor memory. The internal storage circuitry 130 stores a program for implementing ultrasound transmission/reception, a program related to bloodstream imaging mode transition processing described later, determination conditions described later, transition conditions described later, various data, and the like. The programs and various data may be pre-stored in, for example, the internal storage circuitry 130. Alternatively, the programs and various data may be stored and distributed in, for example, a non-transitory storage medium, and read from the non-transitory storage medium and installed in the internal storage circuitry 130. The internal storage circuitry 130 stores B-mode image data, contrast image data, image data related to a bloodstream image, and the like that are generated at the processing circuitry 180, in accordance with an operation that is input via the input interface 150. The internal storage circuitry 130 can also transfer the stored image data to the external device 104 or the like via the communication interface 170.

The internal storage circuitry 130 may be a drive or the like which reads and writes various types of information to and from a portable storage medium, such as a CD drive, a DVD drive, and a flash memory. The internal storage circuitry 130 can also write the stored data onto a portable storage medium and store the data in the external device 104 through the portable storage medium.

The image memory 140 includes, for example, a magnetic storage medium, an optical storage medium, or a processor-readable storage medium such as a semiconductor memory. The image memory 140 stores image data corresponding to a plurality of frames immediately before a freeze operation input via the input interface 150. The image data stored in the image memory 140 is, for example, continuously displayed (cine-displayed).

The internal storage circuitry 130 and the image memory 140 need not necessarily be implemented by independent storage devices. The internal storage circuitry 130 and the image memory 140 may be implemented by a single storage device. Each of the internal storage circuitry 130 and the image memory 140 may be implemented by a plurality of storage devices.

The input interface 150 receives various commands from an operator through the input device 102. The input device 102 is, for example, a mouse, a keyboard, a panel switch, a slider switch, a trackball, a rotary encoder, an operation panel, or a touch command screen (TCS). The input interface 150 is connected to the processing circuitry 180 via a bus, for example, thereby converting an operation command that is input by the operator, into an electric signal, and outputting the electric signal to the processing circuitry 180. The input interface 150 is not limited to a component that is connected to a physical operation component such as a mouse and keyboard. Examples of the input interface also include circuitry that is configured to receive an electric signal corresponding to an operation command input from an external input device provided separately from the ultrasound diagnosis apparatus 1 and to output the electric signal to the processing circuitry 180.

The output interface 160 is an interface for outputting, for example, an electric signal from the processing circuitry 180 to the output device 103. The output device 103 is any display such as a liquid crystal display, an organic EL display, an LED display, a plasma display, or a CRT display. The output device 103 may be a touch panel display that doubles as the input device 102. The output device 103 may further include a speaker that outputs voice in addition to a display. The output interface 160 is connected to the processing circuitry 180 via a bus, for example, and outputs an electric signal from the processing circuitry 180 to the output device 103.

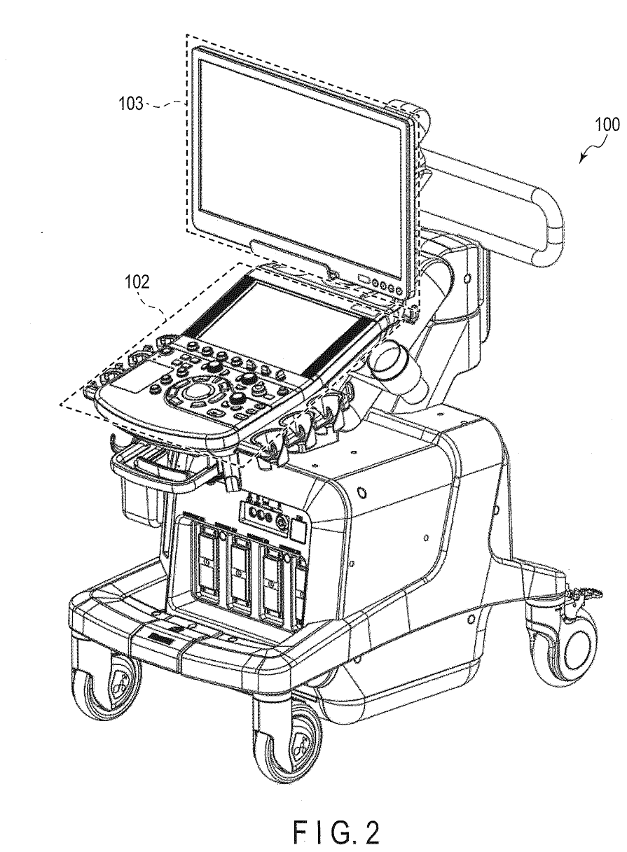

FIG. 2 is a perspective view of an exterior of the apparatus main body of the ultrasound diagnosis apparatus in the first embodiment. In the apparatus main body 100 shown in FIG. 2, the input device 102 and the output device 103 are connected to each other. A user operates the input device 102 and obtains desired clinical information by viewing the output device 103.

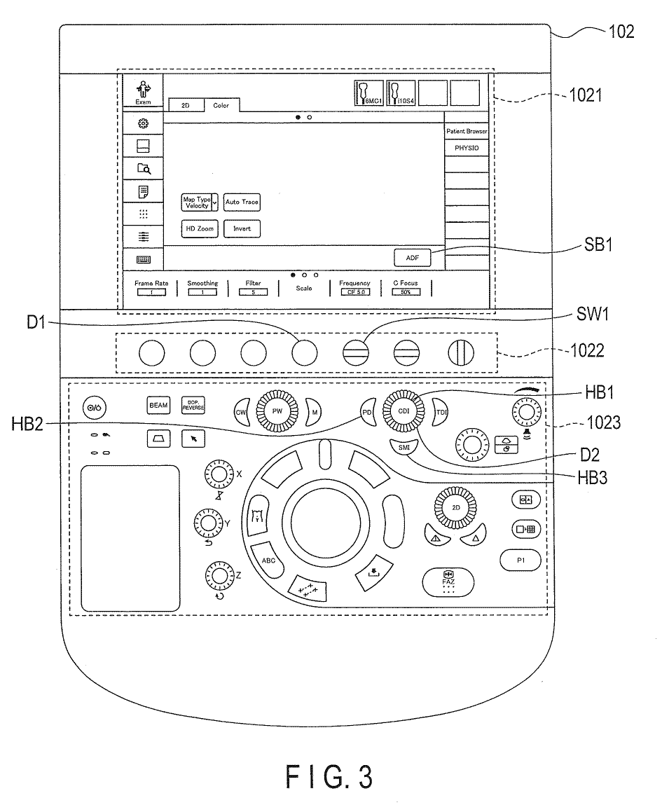

FIG. 3 is a top view of an exterior of the input device connected to the ultrasound diagnosis apparatus in the first embodiment. The input device 102 shown in FIG. 3 includes a touch panel 1021, a first operation unit 1022, and a second operation unit 1023.

For example, a setting screen of the ultrasound diagnosis apparatus is displayed on the touch panel 1021. The setting screen includes a software button for switching the connected ultrasonic probe, a software button for changing to another mode, setting items that can be changed in accordance with the operation of the first operation unit 1022, and the like.

The first operation unit 1022 is configured by, for example, a dial knob, a vertically movable switch, and a horizontally movable switch. The first operation unit 1022 is used, for example, when changing the setting items displayed on the screen of the touch panel 1021.

The second operation unit 1023 is configured by, for example, a dial ring, a hardware button, a wheel, and a trackball. The second operation unit 1023 is used, for example, when directly changing the mode of the ultrasound diagnosis apparatus. The second operation unit 1023 is also used, for example, when changing the parameter (display parameter) related to the display of the ultrasonic image displayed on the display as the output device 103.

The communication interface 170 is connected to the external device 104 via, for example, the network NW, and performs data communication with the external device 104.

The processing circuitry 180 is, for example, a processor that functions as the center of the ultrasound diagnosis apparatus 1. The processing circuitry 180 executes a program stored in the internal storage circuitry 130, thereby implementing a function corresponding to the program. The processing circuitry 180 includes, for example, a B-mode processing function 181, a Doppler processing function 182, an image generation function 183 (image generator), an acquisition function 184 (acquisition unit), a determination function 185 (determination unit), a transition control function 186 (transition controller), a display control function 187 (display controller), and a system control function 188.

The B-mode processing function 181 is a function of generating B-mode data based on the reception signal received from the ultrasound reception circuitry 120. With the B-mode processing function 181, the processing circuitry 180 performs, for example, envelope detection processing, logarithmic compression processing, and the like on the reception signal received from the ultrasonic reception circuitry 120 to generate data (B-mode data) that expresses a signal intensity by brightness. The generated B-mode data is stored in a raw data memory (not shown) as B-mode raw data on a two-dimensional ultrasonic scanning line (raster).

Further, with the B-mode processing function 181, the processing circuitry 180 can execute a contrast echo method such as contrast harmonic imaging (CHI). Specifically, the processing circuitry 180 can separate reflection wave data (a harmonic component or a subharmonic component) of the living body P injected with a contrast agent, and reflection wave data (a fundamental wave component) whose reflection source is a tissue in the living body P. As a result, the processing circuitry 180 can extract a harmonic component or a subharmonic component from the reflection wave data of the living body P, to generate B-mode data for generating contrast image data.

The B-mode data for generating contrast image data is data expressing a signal intensity of the reflection wave, whose reflection source is a contrast agent, by brightness. The processing circuitry 180 can extract a fundamental wave component from the reflection wave data of the living body P, to generate B-mode data for generating tissue image data.

When performing the CHI, the processing circuitry 180 can extract a harmonic component by a method other than the above-described method using the filtering process. In the harmonic imaging, an amplitude modulation (AM) method, a phase modulation (PM) method, or an imaging method called an AMPM method, which is a combination of the AM method and the PM method, is performed.

In the AM method, the PM method, and the AMPM method, ultrasound transmission is performed more than once for a single scanning line, with different amplitudes and/or phases. Through the above processing, the ultrasound reception circuitry 120 generates a plurality of reflection wave data at each scanning line, and outputs the generated reflection wave data. With the B-mode processing function 181, the processing circuitry 180 performs addition/subtraction processing to the plurality of reflection wave data at the respective scanning lines in accordance with a modulation method, thereby extracting a harmonic component. Then, the processing circuitry 180 performs envelope detection processing or the like to the reflection wave data of the harmonic component, thereby generating B-mode data.

The Doppler processing function 182 is a function of analyzing the frequency of the reception signal received from the ultrasonic reception circuitry 120 and thereby generating data (Doppler information) obtained by extracting motion information based on the Doppler effect of a moving object in a region of interest (ROI) set in the scan area. The generated Doppler information is stored in a raw data memory (not shown) as Doppler raw data (also referred to as “Doppler data”) on a two-dimensional ultrasonic scanning line.

Specifically, with the Doppler processing function 182, the processing circuitry 180 estimates an average velocity, an average dispersion value, an average power value, etc., for example, as motion information of a moving object at each sampling point, and generates Doppler data indicating the estimated motion information. The moving object is, for example, a bloodstream, tissue of a cardiac wall, etc., and a contrast agent. With the Doppler processing function 182, the processing circuitry 180 according to the present embodiment estimates an average bloodstream velocity, a dispersion value of a bloodstream velocity, a power value of a bloodstream signal, etc., as motion information of a bloodstream (bloodstream information) at each sampling point, and generates Doppler data indicating the estimated bloodstream information.

Furthermore, with the Doppler processing function 182, the processing circuitry 180 can perform a color Doppler method also called a color flow mapping (CFM) method. In the CFM method, the transmission and reception of ultrasound waves are performed on multiple scanning lines more than once. In the CFM method, a moving target indicator (MTI) filter is applied to data strings in the same position, for example, to thereby suppress signals (clutter signals) related to stationary tissue or slow-moving tissue so that bloodstream-related signals are extracted. In the CFM method, the extracted bloodstream signals are used to estimate the bloodstream information such as a bloodstream velocity, bloodstream dispersion, and bloodstream power. With the image generation function 183 described later, a distribution of the estimated bloodstream information is generated, for example, as ultrasonic image data (color Doppler image data) displayed in color in two dimensions. Hereinafter, the mode of the ultrasound diagnosis apparatus in which bloodstream signals are extracted through an MTI filter based on the Doppler method and the extracted bloodstream signals are used for imaging will be referred to as a “bloodstream imaging mode”. Color display refers to displaying the distribution of the bloodstream information in accordance with a predetermined color code, and includes gray-scale display.

The bloodstream imaging mode includes various types in accordance with desired clinical information. In general, there are a bloodstream imaging mode for velocity display that allows for visualization of a bloodstream direction or an average bloodstream velocity, and a bloodstream imaging mode for power display that allows for visualization of bloodstream signal power.

The bloodstream imaging mode for velocity display is a mode of displaying color corresponding to the Doppler shift frequency based on a bloodstream direction or average bloodstream velocity. For example, the bloodstream imaging mode for velocity display represents, as a flow direction, an oncoming flow by a red-based color and a receding flow by a blue-based color, thereby representing the difference in the velocity between the oncoming flow and the receding flow by the difference in the hue. The bloodstream imaging mode for velocity display may also be called a “color Doppler mode” or a “color Doppler imaging (CDI) mode”. The “bloodstream direction or average bloodstream velocity” may be rephrased as “bloodstream flow rate information”.

The bloodstream imaging mode for power display is a mode of representing bloodstream signal power by, for example, a red-based color phase, brightness of the color, or a change in chromaticness. The bloodstream imaging mode for power display may also be called a “power Doppler (PD) mode”. Since the bloodstream imaging mode for power display can represent a bloodstream at high sensitivity, as compared to the bloodstream imaging mode for velocity display, the bloodstream imaging mode for power display may be called a high-sensitivity bloodstream imaging mode. The “bloodstream signal power” may be rephrased as “bloodstream power information”.

In addition to the CDI mode and the PD mode, there are a bloodstream imaging mode for low flow rate specialized in representing a low flow rate, a high-resolution bloodstream imaging mode, and the like. These four bloodstream imaging modes have different imaging methods defined by a scan protocol, signal processing, and the like. A detailed description of the imaging methods will be given later.

The image generation function 183 is a function of generating B-mode image data based on the data generated by the B-mode processing function 181. With the image generation function 183, the processing circuitry 180, for example, converts (scan-converts) a scanning line signal sequence of an ultrasonic scan into a scanning line signal sequence of a video format representatively used by television, etc. to generate image data for display (display image data). Specifically, the processing circuitry 180 executes a raw-pixel conversion, such as a coordinate conversion corresponding to the mode of the ultrasonic scan by the ultrasonic probe 101, on B-mode raw data stored in the raw data memory to generate two-dimensional B-mode image data (also referred to as “ultrasonic image data”) consisting of pixels. In other words, with the image generation function 183, the processing circuitry 180 generates a plurality of ultrasonic images (medical images) respectively corresponding to a plurality of consecutive frames by transmission and reception of ultrasound waves.

The processing circuitry 180 also executes, for example, a raw-pixel conversion on Doppler raw data stored in the raw data memory to generate Doppler image data that visualizes bloodstream information. The Doppler image data is average velocity image data, dispersion image data, power image data, or image data of a combination thereof. The processing circuitry 180 generates, as Doppler image data, color Doppler image data that represents the bloodstream information by color and Doppler image data that represents a piece of bloodstream information in a waveform shape with a gray scale. The color Doppler image data is generated when the above-described bloodstream imaging mode is executed.

The acquisition function 184 is a function of acquiring a parameter (display parameter) related to image display input by a user. With the acquisition function 184, the processing circuitry 180, for example, receives input (change) of the display parameter from the user. In other words, with the acquisition function 184, the processing circuitry 180 acquires the change in the display parameter related to the image display in the current bloodstream imaging mode.

The determination function 185 is a function of determining whether or not the acquired display parameter satisfies a predetermined condition (transition condition). In other words, with the determination function 185, the processing circuitry 180 determines whether or not the display parameter after the change is more suitable for another bloodstream imaging mode than for the current bloodstream imaging mode. Specifically, with the determination function 185, the processing circuitry 180 reads the transition condition stored in the internal storage circuitry 130, and determines whether or not the acquired display parameter satisfies a predetermined condition.

The predetermined condition may be, for example, whether or not the display parameter is equal to or greater than a threshold, or whether or not the display parameter is less than a threshold. Alternatively, the predetermined condition may be, for example, whether or not a plurality of display parameters are equal to or greater than a threshold corresponding to the respective parameters, or whether or not a plurality of display parameters are less than a threshold corresponding to the respective parameters. The predetermined condition may also be, for example, whether or not one of a plurality of parameters is equal to or greater than a threshold and whether or not the other parameter is less than a threshold, and vice versa. The predetermined condition may be based on another parameter changed according to the display parameter.

The transition control function 186 is a function of transitioning to a recommended bloodstream imaging mode based on the parameter after the change when the determination function 185 determines that the predetermined condition is satisfied. In other words, with the transition control function 186, the processing circuitry 180 transitions from the current bloodstream imaging mode to another bloodstream imaging mode when the display parameter after the change is more suitable for another bloodstream imaging mode than for the current bloodstream imaging mode. Specifically, with the transition control function 186, the processing circuitry 180 reads the transition condition stored in the internal storage circuitry 130, and transitions to a bloodstream imaging mode corresponding to the predetermined condition satisfied at the time of the determination. For the transition condition, for example, a predetermined condition and a recommended bloodstream imaging mode are associated with each other. That is, the display parameter, threshold, and bloodstream imaging mode to which a transition is made are associated with one another. For the transition condition, a bloodstream imaging mode prior to transition may be further associated therewith. The threshold of the transition condition may be changed by the user.

The display control function 187 is a function of causing a display as the output device 103 to display an image based on various kinds of ultrasonic image data generated by the image generation function 183. Specifically, with the display control function 187, the processing circuitry 180, for example, controls the displaying, on the display, of an image based on the B-mode image data, the Doppler image data, or the image data including both of these types of data, generated by the image generation function 183.

With the display control function 187, the processing circuitry 180 displays information on the transition of the bloodstream imaging mode. Specifically, the processing circuitry 180 performs displaying for notifying the user that a transition will be made or performs displaying for notifying the user that a transition has been made.

More specifically, with the display control function 187, the processing circuitry 180 converts (scan-converts) a scanning line signal sequence of an ultrasonic scan into a scanning line signal sequence of a video format representatively used by television, etc., to generate display image data. The processing circuitry 180 may also perform various types of processing, such as dynamic range, brightness, contrast, y curve corrections, and an RGB conversion, on the display image data. The processing circuitry 180 may also add supplementary information, such as textual information of various parameters, a scale, or a body mark, to the display image data. The processing circuitry 180 may also generate a user interface (graphical user interface (GUI)) to allow the operator to input various commands through the input device, and cause the display to display the GUI.

The system control function 188 is a function of integrally controlling the overall operations of the ultrasound diagnosis apparatus 1. For example, with the system control function 188, the processing circuitry 180 controls the ultrasound transmission circuitry 110 and the ultrasound reception circuitry 120 based on a parameter related to transmission and reception of ultrasound waves.

Next, an operation performed by a user will be concretely described with reference to FIG. 3. To select a desired bloodstream imaging mode before starting examination, a user needs to, for example, press a hardware button HB1, a hardware button HB2, and a hardware button HB3 of the second operation unit 1023. These buttons are associated with different bloodstream imaging modes, respectively. In FIG. 3, for example, the hardware button HB1 is associated with the CDI mode, the hardware button HB2 is associated with the PD mode, and the hardware button HB3 is associated with the bloodstream imaging mode for low flow rate.

Selection of the bloodstream imaging modes is not necessarily made through the hardware buttons. For example, a high-resolution bloodstream imaging mode is executed by further selecting a software button SB1 displayed on the touch panel 1021 in a state where the CDI mode has been selected.

Also, to change the display parameter of the ultrasonic image displayed on the display, the user needs to, for example, operate a knob D1 and a switch SW1 of the first operation unit 1022 and a ring D2 of the second operation unit 1023.

Examples of the display parameter include a flow rate range indicating the upper limit of the display of the average bloodstream velocity, a transmission frequency, and a color gain for adjusting a signal range of displaying the color Doppler image data on the display. In FIG. 3, for example, the operation of the knob D1 is associated with a change of the flow rate range, the operation of the switch SW1 is associated with a change of the transmission frequency, and the operation of the ring D2 is associated with a change of the color gain.

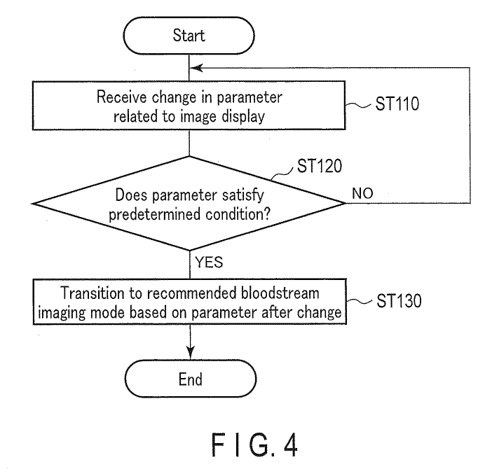

FIG. 4 is a flowchart for explaining operations of the processing circuitry for executing the bloodstream imaging mode transition processing in the first embodiment. The bloodstream imaging mode transition processing in the first embodiment is processing of automatically transitioning from a currently executed bloodstream imaging mode to another bloodstream imaging mode (recommended bloodstream imaging mode). The bloodstream imaging mode transition processing shown in FIG. 4 is started, for example, when the user executes any bloodstream imaging mode.

### (Step ST110)

When the bloodstream imaging mode transition processing starts, the processing circuitry 180 executes the acquisition function 184. When the processing circuitry 180 executes the acquisition function 184, the processing circuitry 180 receives the change in the parameter (display parameter) related to the image display by the user. At this time, the user changes at least one of the flow rate range, transmission frequency, or color gain, for example, in order to change the display parameter of the ultrasonic image displayed on the display.

### (Step ST120)

After receiving the change in the display parameter from the user, the processing circuitry 180 executes the determination function 185. When the processing circuitry 180 executes the determination function 185, the processing circuitry 180 determines whether or not the changed parameter satisfies a predetermined condition.

For example, the processing circuitry 180 determines whether or not the changed value of the display parameter is equal to or greater than a threshold. When the display parameter satisfies a predetermined condition (when the changed value is equal to or greater than a threshold), the processing proceeds to step ST130, and when the display parameter does not satisfy a predetermined condition (when the changed value is less than a threshold), the processing returns to step ST110.

### (Step ST130)

After determining that the display parameter satisfies a predetermined condition, the processing circuitry 180 executes the transition control function 186. When the processing circuitry 180 executes the transition control function 186, the processing circuitry 180 transitions to a recommended bloodstream imaging mode based on the parameter after the change. At this time, the recommended bloodstream imaging mode is associated with the parameter after the change. After step ST130, the processing is completed. The processing may return to step ST110 after step ST130.

When transitioning to the recommended bloodstream imaging mode in step ST130, the processing circuitry 180 through the display control function 187 may perform, on the display, displaying for notifying the user that a transition will be made or displaying for notifying the user that a transition has been made.

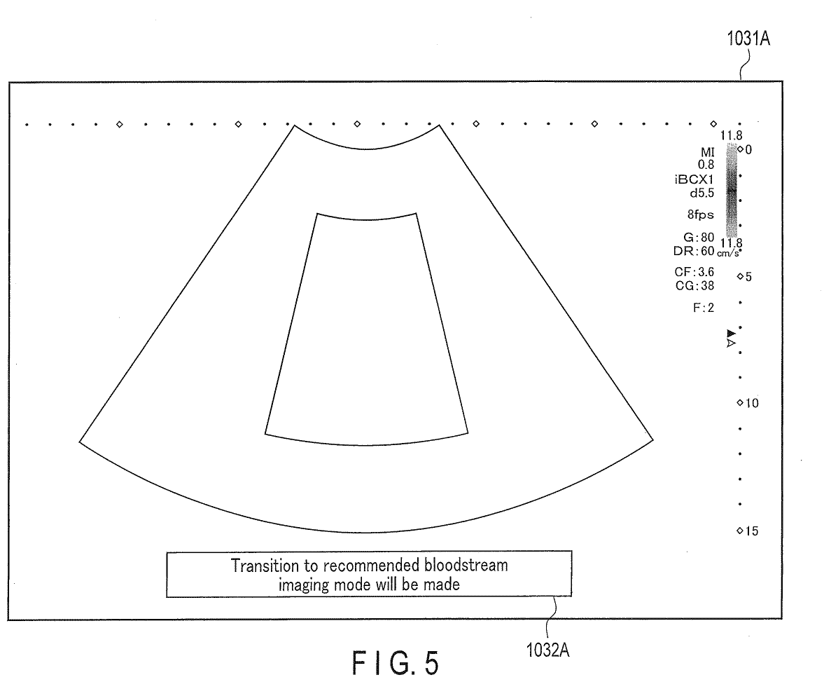

FIG. 5 is a diagram illustrating a display image including a display example for giving notice of a transition to a recommended bloodstream imaging mode in the first embodiment. FIG. 5 shows a display image 1031A displayed on the output device 103 as a display. The display image 1031A includes an ultrasonic image and a notification box 1032A displayed on the ultrasonic image. For example, a text “A transition to a recommended bloodstream imaging mode will be made” is displayed in the notification box 1032A. The notification box 1032A is displayed, for example, at a position not overlapping with the ROI on the ultrasonic image.

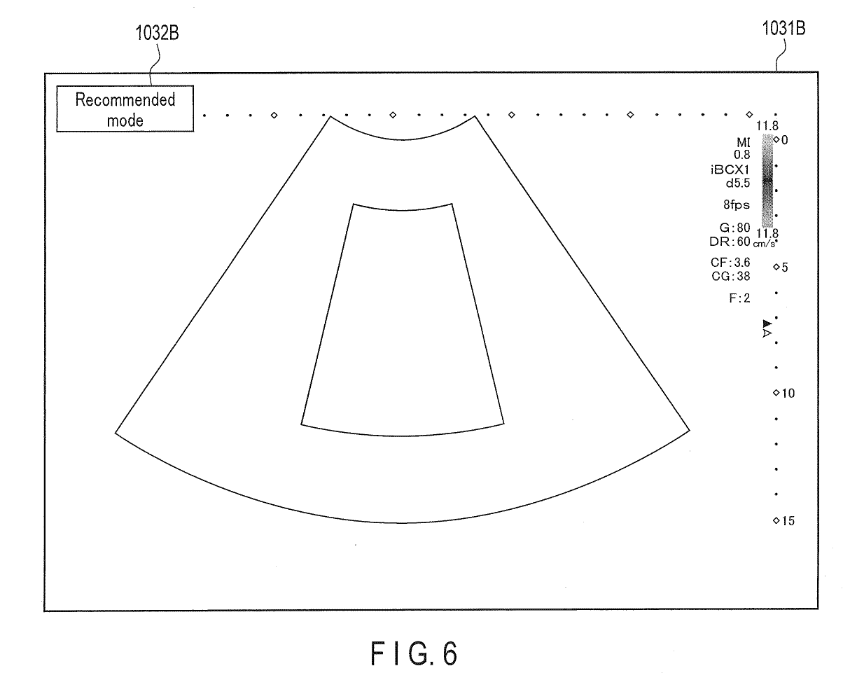

FIG. 6 is a diagram illustrating a display image including a display example showing that a transition to a recommended bloodstream imaging mode has been made in the first embodiment. FIG. 6 shows a display image 1031B displayed on the output device 103 as a display. The display image 1031B includes an ultrasonic image and a notification box 1032B displayed on the ultrasonic image. For example, a text “Recommended Mode” is displayed in the notification box 1032B. The notification box 1032B is displayed, for example, at a position not overlapping with the ROI on the ultrasonic image.

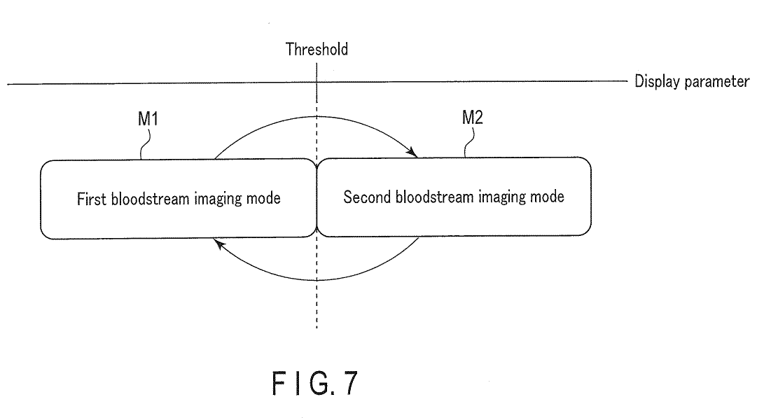

FIG. 7 is a diagram for explaining the bloodstream imaging mode transition processing in the first embodiment. In FIG. 7, a second bloodstream imaging mode M2 may be associated with the display parameter, a first bloodstream imaging mode M1 may be associated with the display parameter, or both the second bloodstream imaging mode M2 and the first bloodstream imaging mode M1 may be associated with the display parameter.

For example, when the current bloodstream imaging mode is the first bloodstream imaging mode M1, and the second bloodstream imaging mode M2 and the display parameter are associated with each other, the processing circuitry 180 transitions from the first bloodstream imaging mode M1 to the second bloodstream imaging mode M2 upon a change of the display parameter to equal to or greater than a threshold. Namely, when the display parameter is equal to or greater than a threshold, the second bloodstream imaging mode M2 is the recommended bloodstream imaging mode, as opposed to the first bloodstream imaging mode M1.

For example, when the current bloodstream imaging mode is the second bloodstream imaging mode M2, and the first bloodstream imaging mode M1 and the display parameter are associated with each other, the processing circuitry 180 transitions from the second bloodstream imaging mode M2 to the first bloodstream imaging mode M1 upon a change of the display parameter to less than a threshold. Namely, when the display parameter is less than a threshold, the first bloodstream imaging mode M1 is the recommended bloodstream imaging mode, as opposed to the second bloodstream imaging mode M2.

For example, when both the first bloodstream imaging mode M1 and the second bloodstream imaging mode M2 are associated with the display parameter, the processing circuitry 180 transitions from the first bloodstream imaging mode M1 to the second bloodstream imaging mode M2 if the display parameter is equal to or greater than a threshold, and the processing circuitry 180 transitions from the second bloodstream imaging mode M2 to the first bloodstream imaging mode M1 if the display parameter is less than a threshold. At this time, when the display parameter is equal to or greater than a threshold, the second bloodstream imaging mode M2 is the recommended bloodstream imaging mode, as opposed to the first bloodstream imaging mode M1, and when the display parameter is less than a threshold, the first bloodstream imaging mode M1 is the recommended bloodstream imaging mode, as opposed to the second bloodstream imaging mode M2.

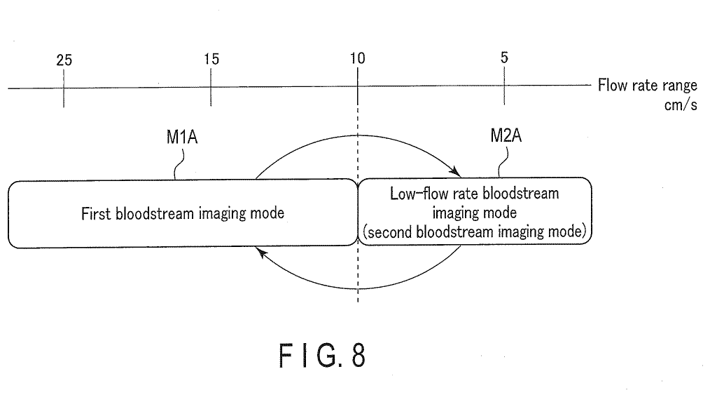

FIG. 8 is a diagram for describing a first specific example of the bloodstream imaging mode transition processing in the first embodiment. In FIG. 8, a bloodstream imaging mode for low flow rate and a flow rate range are associated with each other. Specifically, when the current bloodstream imaging mode is a first bloodstream imaging mode MIA, the processing circuitry 180 transitions from the first bloodstream imaging mode M1A to a low-flow rate bloodstream imaging mode M2A upon a change of the flow rate range to less than 10 cm/s, for example.

In the low-flow rate bloodstream imaging mode M2A after the transition, the processing circuitry 180 may transition from the low-flow rate bloodstream imaging mode M2A to the original first bloodstream imaging mode M1A upon a change of the flow rate range to 10 cm/s or more. The processing circuitry 180 may also make a transition of the mode based on a pulse repetition frequency changed according to the flow rate range. That is, the processing circuitry 180 may compare the PRF changed by changing the flow rate range with a threshold.

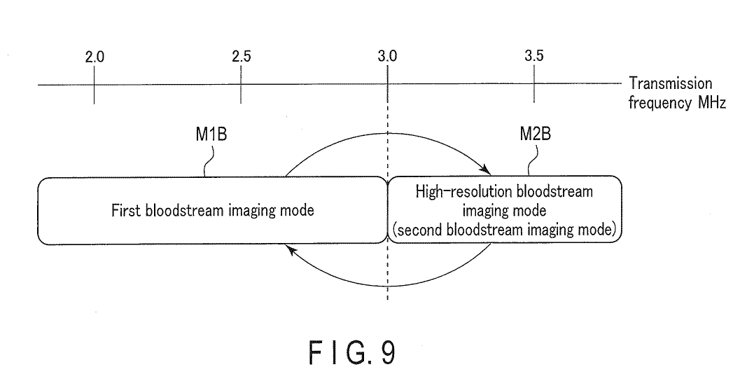

FIG. 9 is a diagram for describing a second specific example of the bloodstream imaging mode transition processing in the first embodiment. In FIG. 9, a high-resolution bloodstream imaging mode and a transmission frequency are associated with each other. Specifically, when the current bloodstream imaging mode is a first bloodstream imaging mode M1B, the processing circuitry 180 transitions from the first bloodstream imaging mode M1B to a high-resolution bloodstream imaging mode M2B upon a change of the transmission frequency to 3.0 MHz or more, for example.

In the high-resolution bloodstream imaging mode M2B after the transition, the processing circuitry 180 may transition from the high-resolution bloodstream imaging mode M2B to the original first bloodstream imaging mode M1B upon a change of the transmission frequency to less than 3.0 MHz.

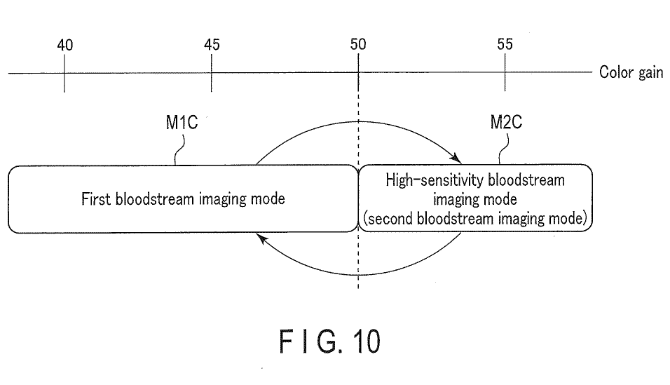

FIG. 10 is a diagram for describing a third specific example of the bloodstream imaging mode transition processing in the first embodiment. In FIG. 10, a high-sensitivity bloodstream imaging mode and a color gain are associated with each other. Specifically, when the current bloodstream imaging mode is a first bloodstream imaging mode MIC, the processing circuitry 180 transitions from the first bloodstream imaging mode MIC to a high-sensitivity bloodstream imaging mode M2C upon a change of the color gain to 50 or more, for example.

In the high-sensitivity bloodstream imaging mode M2C after the transition, the processing circuitry 180 may transition from the high-sensitivity bloodstream imaging mode M2C to the original first bloodstream imaging mode M1C upon a change of the color gain to less than 50.

In the above-described specific examples shown in FIGS. 8 to 10, the first bloodstream imaging mode is not particularly limited. However, the first bloodstream imaging mode and the bloodstream imaging mode after the transition may be associated with each other.

As described above, the ultrasound diagnosis apparatus according to the first embodiment acquires a change in a display parameter related to image display in the first bloodstream imaging mode during execution of the first bloodstream imaging mode of displaying a bloodstream signal acquired by the ultrasonic probe, and determines whether or not the display parameter after the change is more suitable for the second bloodstream imaging mode, which differs from the first bloodstream imaging mode in its imaging method, than for the first bloodstream imaging mode. The present ultrasound diagnosis apparatus transitions from the first bloodstream imaging mode to the second bloodstream imaging mode when the display parameter after the change is more suitable for the second bloodstream imaging mode than for the first bloodstream imaging mode.

Therefore, the ultrasound diagnosis apparatus according to the first embodiment can easily make proper use of different bloodstream images by transitioning to a recommended bloodstream imaging mode according to the operation by the user.

Also, the ultrasound diagnosis apparatus according to the first embodiment can display information indicating that a transition will be made from the first bloodstream imaging mode to the second bloodstream imaging mode. Thus, the user can know that a transition to another mode will be made.

In addition, the ultrasound diagnosis apparatus according to the first embodiment can display information indicating that a transition has been made from the first bloodstream imaging mode to the second bloodstream imaging mode. Thus, the user can know that a transition to another mode has been made.

### Application Example of First Embodiment

In the first embodiment, an automatic transition of the bloodstream imaging mode is described. On the other hand, in an application example of the first embodiment, a transition of the bloodstream imaging mode based on a transition command by a user will be described.

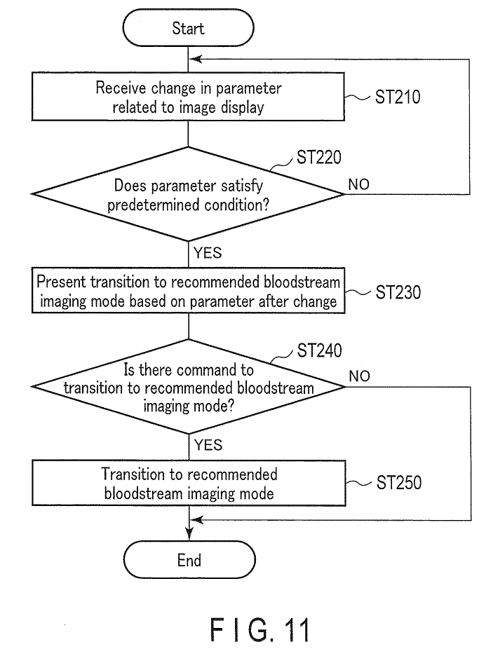

FIG. 11 is a flowchart for explaining operations of processing circuitry that executes bloodstream imaging mode transition processing in the application example of the first embodiment. The bloodstream imaging mode transition processing in the application example of the first embodiment is processing of presenting a user with a transition from a currently executed bloodstream imaging mode to another bloodstream imaging mode (recommended bloodstream imaging mode) and making the transition based on the user's command. The bloodstream imaging mode transition processing shown in FIG. 11 is started, for example, when the user executes any bloodstream imaging mode. Since steps ST210 and ST220 are the same as the above-described steps ST110 and ST120, respectively, descriptions thereof will be omitted.

### (Step ST230)

After determining that the display parameter satisfies a predetermined condition, the processing circuitry 180 executes the display control function 187. When the processing circuitry 180 executes the display control function 187, the processing circuitry 180 presents a transition to a recommended bloodstream imaging mode based on the parameter after the change. At this time, the recommended bloodstream imaging mode is associated with the parameter after the change.

For example, there are two methods of presenting a transition to a recommended bloodstream imaging mode. One of them is to perform displaying for prompting a transition to a recommended bloodstream imaging mode, wherein a software button on the screen of the touch panel is selected when the transition is actually made. The other one is to display a transition to a recommended bloodstream imaging mode together with an option.

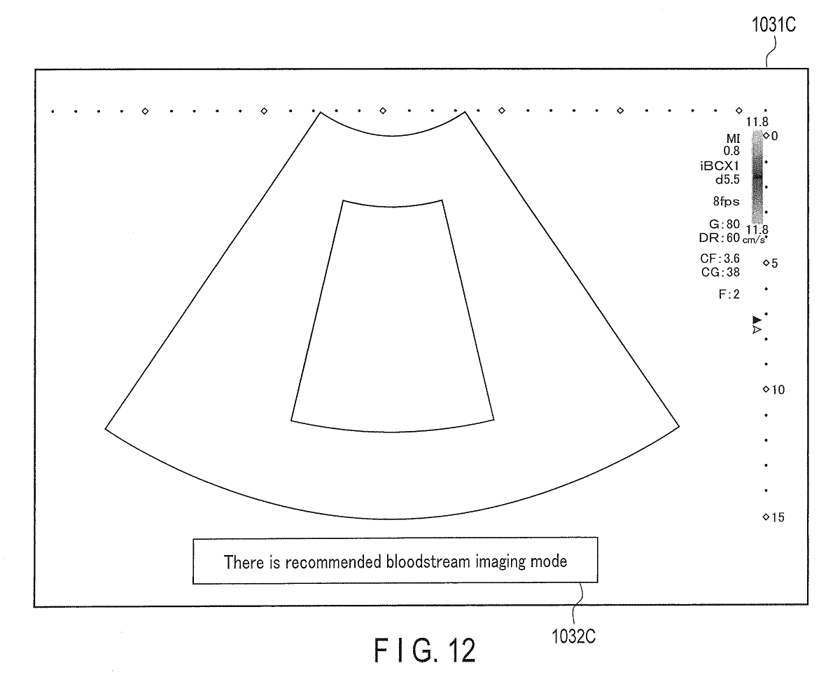

FIG. 12 is a diagram illustrating a display image including a display example for prompting a transition to a recommended bloodstream imaging mode in the application example of the first embodiment. FIG. 12 shows a display image 1031C displayed on the output device 103 as a display. The display image 1031C includes an ultrasonic image and a notification box 1032C displayed on the ultrasonic image. For example, a text “There is a recommended bloodstream imaging mode” is displayed in the notification box 1032C. The notification box 1032C is displayed, for example, at a position not overlapping with the ROI on the ultrasonic image.

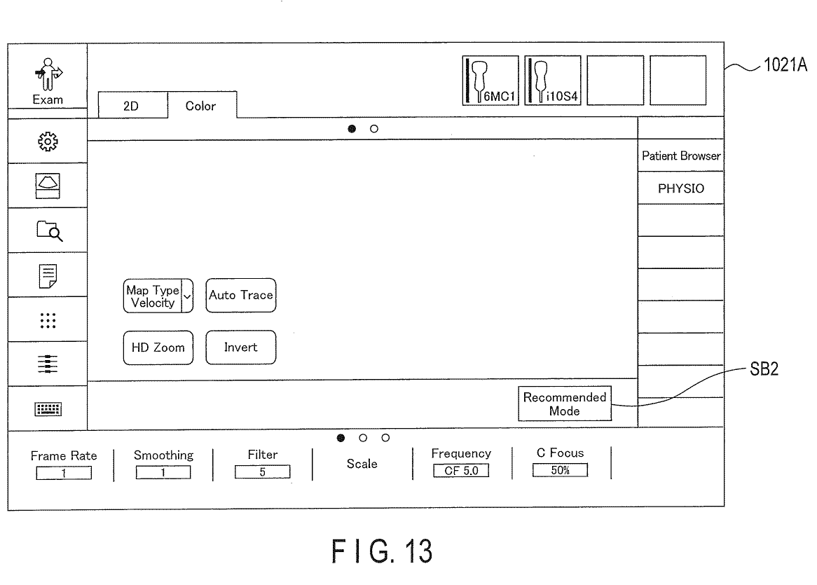

FIG. 13 is a diagram illustrating a display image of a touch panel including a software button for transitioning to a recommended bloodstream imaging mode in the application example of the first embodiment. A touch panel 1021A shown in FIG. 13 displays a software button SB2 for transitioning to a recommended bloodstream imaging mode. For example, a text “Recommended Mode” is displayed on the software button SB2 to prompt selection by the user. When the user selects the software button SB2, a transition to a recommended bloodstream imaging mode is made according to the user's selection as a transition command. To prompt selection by the user, the software button SB2 may have a display color different from those of the other buttons or may blink.

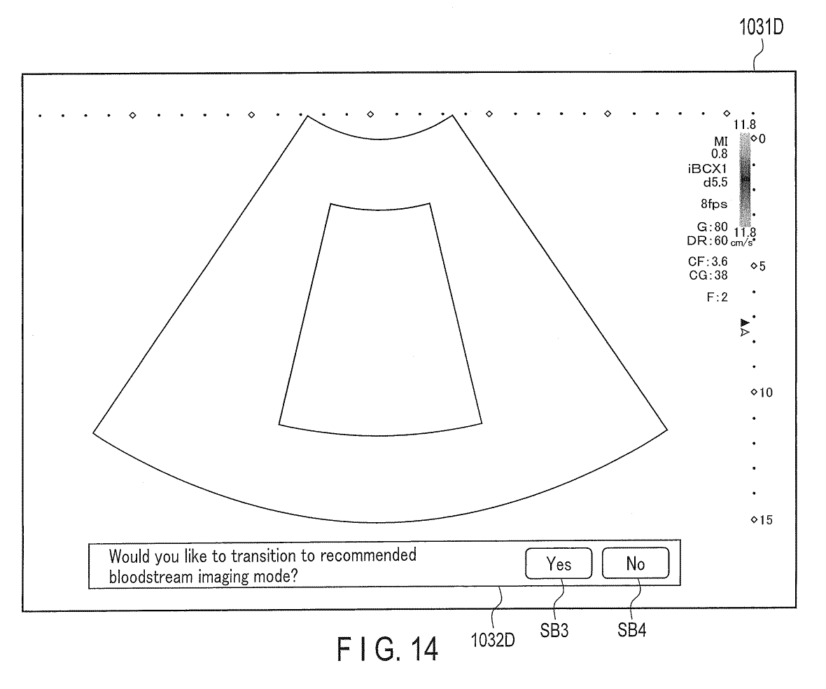

FIG. 14 is a diagram illustrating a display image including a display example for prompting a transition to a recommended bloodstream imaging mode, and options in the application example of the first embodiment. FIG. 14 shows a display image 1031D displayed on the output device 103 as a display. The display image 1031D includes an ultrasonic image and a notification box 1032D displayed on the ultrasonic image. For example, a text “Would you like to transition to a recommended bloodstream imaging mode?”, the software button SB3, and the software button SB4 are displayed in the notification box 1032D. Indications “Yes” and “No” are displayed on the software button SB3 and the software button SB4, respectively. When the user selects the software button SB3 “Yes”, a transition to a recommended bloodstream imaging mode is made according to the user's selection as a transition command, and when the user selects the software button SB4 “No”, no transition is made with the current bloodstream imaging mode maintained.

### (Step ST240)

After presenting the user with a transition to a recommended bloodstream imaging mode, the processing circuitry 180 waits for a transition command from the user. When the transition command is received from the user, the processing proceeds to step ST250, and when no transition command is received from the user, or when a command to not make a transition is received, the processing is completed.

### (Step ST250)

After receiving the transition command from the user, the processing circuitry 180 executes a transition control function 186. When the processing circuitry 180 executes the transition control function 186, the processing circuitry 180 transitions to a recommended bloodstream imaging mode. After step ST250, the processing is completed. The processing may return to step ST210 after step ST250.

When transitioning to the recommended bloodstream imaging mode in step ST250, the processing circuitry 180 through the display control function 187 may perform, on the display, displaying for notifying the user that a transition will be made or displaying for notifying the user that a transition has been made. Said displaying is the same as those shown in FIGS. 5 and 6, for example.

As described above, the ultrasound diagnosis apparatus according to the application example of the first embodiment acquires a change in a display parameter related to image display in the first bloodstream imaging mode during execution of the first bloodstream imaging mode of displaying a bloodstream signal acquired by the ultrasonic probe, and determines whether or not the display parameter after the change is more suitable for the second bloodstream imaging mode differing from the first bloodstream imaging mode in the imaging method than for the first bloodstream imaging mode. The present ultrasound diagnosis apparatus displays information that recommends the second bloodstream imaging mode when the display parameter after the change is more suitable for the second bloodstream imaging mode than for the first bloodstream imaging mode.

Therefore, the ultrasound diagnosis apparatus according to the application example of the first embodiment can easily make proper use of different bloodstream images owing to the presentation of the recommended bloodstream imaging mode according to the operation by the user.

Also, the ultrasound diagnosis apparatus according to the application example of the first embodiment can display information indicating whether or not to make a transition to the second bloodstream imaging mode. The present ultrasound diagnosis apparatus can transition from the first bloodstream imaging mode to the second bloodstream imaging mode when a transition command is issued. Thus, the user can know that a transition to another mode can be made, so that the user can select whether or not to make a transition.

In addition, the ultrasound diagnosis apparatus according to the application example of the first embodiment can display information indicating that a transition has been made from the first bloodstream imaging mode to the second bloodstream imaging mode. Thus, the user can know that a transition to another mode has been made.

### Second Embodiment

In the first embodiment and the application example of the first embodiment, a transition between the bloodstream imaging modes is described. On the other hand, in the second embodiment, a transition between the bloodstream imaging methods in the same bloodstream imaging mode will be described.

In the first embodiment and the application example of the first embodiment, one bloodstream imaging mode is associated with one bloodstream imaging method. For example, the bloodstream imaging mode for velocity display is associated with a bloodstream imaging method for velocity display, and the bloodstream imaging mode for power display is associated with a bloodstream imaging method for power display. The same applies to other bloodstream imaging modes.

On the other hand, in the second embodiment, a single bloodstream imaging mode includes a plurality of bloodstream imaging methods differing in the imaging method. Specifically, a specific bloodstream imaging mode, for example, includes a bloodstream imaging method for velocity display and a bloodstream imaging method for power display, and another specific bloodstream imaging mode includes a low-flow rate bloodstream imaging method and a high-resolution bloodstream imaging method.

The bloodstream imaging methods are defined by a scan protocol and signal processing. Therefore, between the different bloodstream imaging methods, at least one of a scan protocol or signal processing differs. Specifically, a drive signal for ultrasonic transmission, an ultrasonic scan method, a wall filter method, a display image processing method, a signal processing method, and the like differ for each bloodstream imaging method.

The drive signal for ultrasonic transmission specifies a frequency, an output intensity, and the like of an ultrasound wave used for transmission. For example, in the bloodstream imaging method for power display, a drive signal is set to have a low frequency and a narrow band, and in the low-flow rate bloodstream imaging method, a drive signal is set to have a high frequency and a wide band.

The ultrasonic scan method specifies a scan timing of an ultrasound wave, and the like. For example, when acquiring B-mode image data for a background and color Doppler image data in an ROI, a scan timing is set so as to decrease the frequency of acquiring the B-mode image data in order to give high place to a frame rate of the color Doppler image data.

The wall filter method specifies a range of applying a wall filter, and the like. The wall filter is the same as the MTI filter described above. In the case of the bloodstream imaging method for velocity display, for example, a filter is set so as to cut a signal of a slow-moving bloodstream in order to selectively display a fast-moving bloodstream.

The display image processing method specifies, for example, a filter applied to a display image. In the case of the bloodstream imaging method for power display, for example, a smoothing filter and the like are set for the purpose of canceling noise.

The signal processing method specifies, for example, processing of an acquired ultrasonic signal. In the case of the low-flow rate bloodstream imaging method, for example, processing of separating a low flow rate bloodstream overlapping with a motion of a body tissue by analyzing the characteristics of the motion of the body tissue is set.

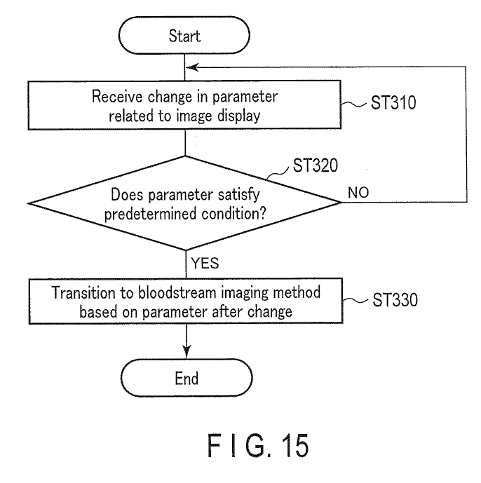

FIG. 15 is a flowchart for explaining operations of the processing circuitry that executes bloodstream imaging method transition processing in the second embodiment. The bloodstream imaging method transition processing is processing of automatically switching (transitioning) from a currently executed bloodstream imaging method to another bloodstream imaging method. The bloodstream imaging method transition processing shown in FIG. 15 is started, for example, when the user executes a specific bloodstream imaging mode. Since steps ST310 and ST320 are the same as above-described steps ST110 and ST120, respectively, descriptions thereof will be omitted.

### (Step ST330)

After determining that the display parameter satisfies a predetermined condition, the processing circuitry 180 executes the transition control function 186. When the processing circuitry 180 executes the transition control function 186, the processing circuitry 180 transitions to another bloodstream imaging method based on the parameter after the change. On this occasion, the bloodstream imaging method after the transition is associated with the changed parameter and the current bloodstream imaging method. After step ST330, the processing is completed. The processing may return to step ST310 after step ST330.

When transitioning to another bloodstream imaging method in step ST330, the processing circuitry 180 through the display control function 187 may perform, on the display, displaying for notifying the user that a transition will be made or displaying for notifying the user that a transition has been made.

After the determination in step ST330, the processing circuitry 180 through the display control function 187 may present a transition to another bloodstream imaging method based on the parameter after the change. In the method of presenting a transition, displaying for prompting a transition may be performed, or a transition may be displayed together with an option, in the same manner as described in the application example of the first embodiment.

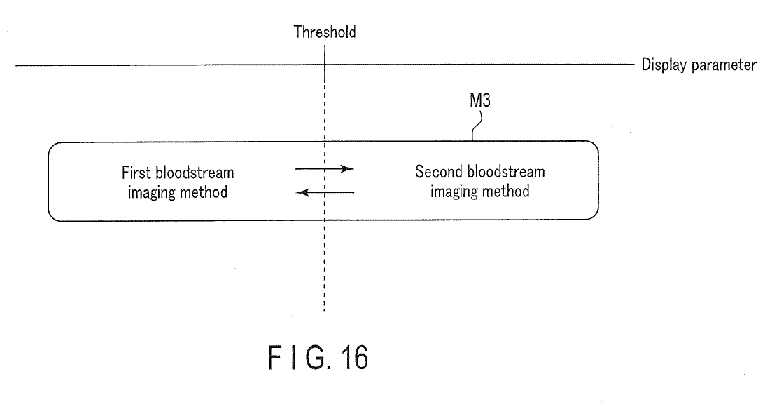

FIG. 16 is a diagram for explaining the bloodstream imaging method transition processing in the second embodiment. In FIG. 16, a first bloodstream imaging method, a second bloodstream imaging method, and a display parameter are associated with one another in a specific bloodstream imaging mode M3.

For example, when displaying is performed using the first bloodstream imaging method as the current bloodstream imaging method, the processing circuitry 180 transitions from the first bloodstream imaging method to the second bloodstream imaging method upon a change of the display parameter to equal to or greater than a threshold. For example, when displaying is performed using the second bloodstream imaging method as the current bloodstream imaging method, the processing circuitry 180 transitions from the second bloodstream imaging method to the first bloodstream imaging method upon a change of the display parameter to less than a threshold. There may be a plurality of display parameters.

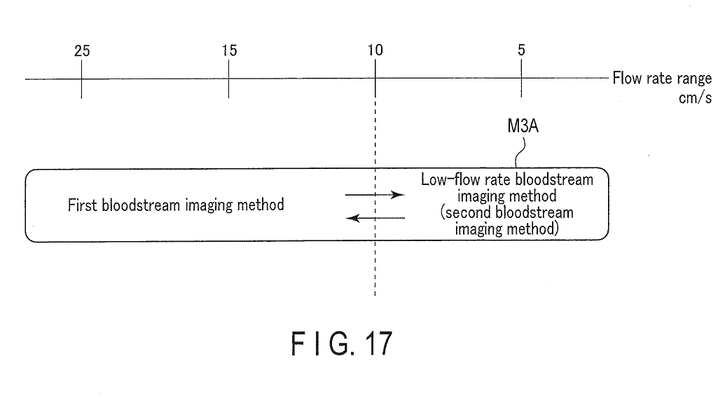

FIG. 17 is a diagram for describing a first specific example of the bloodstream imaging method transition processing in the second embodiment. FIG. 17 shows a bloodstream imaging mode M3A in which a first bloodstream imaging method, a low-flow rate bloodstream imaging method, and a flow rate range are associated with one another. Specifically, when displaying is performed using the first bloodstream imaging method as the current bloodstream imaging method, the processing circuitry 180 transitions from the first bloodstream imaging method to the low-flow rate bloodstream imaging method upon a change of the flow rate range to less than 10 cm/s, for example. Also, in the low-flow rate bloodstream imaging method after the transition, the processing circuitry 180 transitions from the low-flow rate bloodstream imaging method to the first bloodstream imaging method upon a change of the flow rate range to 10 cm/s or more.

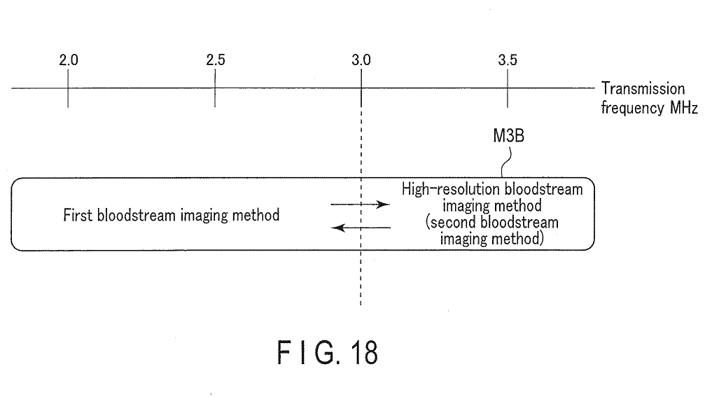

FIG. 18 is a diagram for describing a second specific example of the bloodstream imaging method transition processing in the second embodiment. FIG. 18 shows a bloodstream imaging mode M3B in which a first bloodstream imaging method, a high-resolution bloodstream imaging method, and a transmission frequency are associated with one another. Specifically, when displaying is performed using the first bloodstream imaging method as the current bloodstream imaging method, the processing circuitry 180 transitions from the first bloodstream imaging method to the high-resolution bloodstream imaging method upon a change of the transmission frequency to 3.0 MHz or more, for example. Also, in the high-resolution bloodstream imaging method after the transition, the processing circuitry 180 transitions from the high-resolution bloodstream imaging method to the first bloodstream imaging method upon a change of the transmission frequency to less than 3.0 MHz.

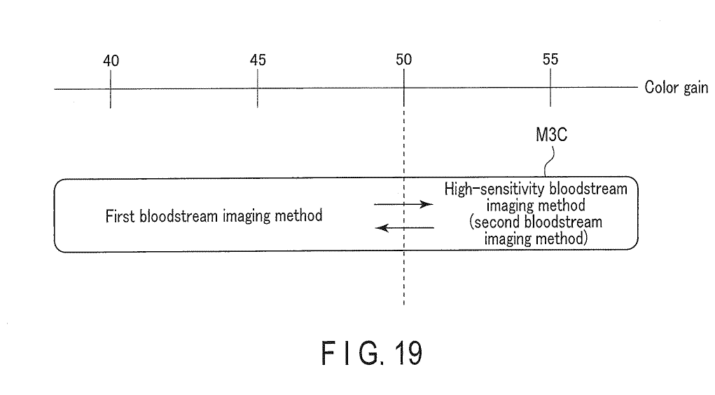

FIG. 19 is a diagram for describing a third specific example of the bloodstream imaging method transition processing in the second embodiment. FIG. 19 shows a bloodstream imaging mode M3C in which a first bloodstream imaging method, a high-sensitivity bloodstream imaging method, and a color gain are associated with one another. Specifically, when displaying is performed using the first bloodstream imaging method as the current bloodstream imaging method, the processing circuitry 180 transitions from the first bloodstream imaging method to the high-sensitivity bloodstream imaging method upon a change of the color gain to 50 or more, for example. Also, in the high-sensitivity bloodstream imaging method after the transition, the processing circuitry 180 transitions from the high-sensitivity bloodstream imaging method to the first bloodstream imaging method upon a change of the color gain to less than 50.

In the above-described specific examples shown in FIGS. 17 to 19, the first bloodstream imaging method is not particularly limited.

As described above, the ultrasound diagnosis apparatus according to the second embodiment acquires a change in the display parameter related to image display in a first bloodstream imaging method during execution of the first bloodstream imaging method, and determines whether or not the display parameter after the change satisfies a predetermined condition, wherein the ultrasound diagnosis apparatus includes a bloodstream imaging mode of displaying a bloodstream signal acquired by the ultrasonic probe, and the bloodstream imaging mode includes the first bloodstream imaging method and a second bloodstream imaging method differing from each other in at least one of a scan protocol or signal processing. The present ultrasound diagnosis apparatus transitions from the first bloodstream imaging method to the second bloodstream imaging method when the predetermined condition is satisfied.

Therefore, the ultrasound diagnosis apparatus according to the second embodiment can easily make proper use of different bloodstream images by transitioning between the bloodstream imaging methods according to the operation by the user.

Also, the ultrasound diagnosis apparatus according to the second embodiment can display information indicating that a transition to the second bloodstream imaging method will be made. Thus, the user can know that a transition to another bloodstream imaging method will be made.

The ultrasound diagnosis apparatus according to the second embodiment can also display information indicating whether or not to make a transition to the second bloodstream imaging method. The present ultrasound diagnosis apparatus can transition from the first bloodstream imaging method to the second bloodstream imaging method when a transition command is issued. Thus, the user can know that a transition to another system can be made, so that the user can select whether or not to make a transition.

The ultrasound diagnosis apparatus according to the second embodiment can also display information indicating that a transition to the second bloodstream imaging method has been made. Thus, the user can know that a transition to another system has been made.

According to at least one of the above-described embodiments, it is possible to easily make proper use of different bloodstream images.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An ultrasound diagnosis apparatus comprising:
processing circuitry configured to:
execute a bloodstream imaging mode that displays a two-dimensional blood flow image based on a blood flow signal acquired by an ultrasound probe, the bloodstream imaging mode including (1) a first bloodstream imaging method, (2) a low-flow-rate bloodstream imaging method different from the first bloodstream imaging method and corresponding to imaging low-flow-rate bloodstream, (3) a velocity bloodstream imaging method, and (4) a power Doppler imaging method;
acquire a flow rate range changed by a user during an execution of the first bloodstream imaging method;
compare the changed flow rate range with a threshold value and determine whether or not the changed flow rate range is less than the threshold value;
cause a first transition from the first bloodstream imaging method to the low-flow-rate bloodstream imaging method in the case where the changed flow rate range is less than the threshold value; and
cause a second transition from the low-flow-rate bloodstream imaging method to the first bloodstream imaging method in the case where the flow rate range is changed to the threshold value or more in the low-flow-rate bloodstream imaging method after the first transition.

2. The ultrasound diagnosis apparatus according to claim 1, wherein the processing circuitry is further configured to display, as text on a display screen, information indicating that the switch was made from the bloodstream imaging mode to the low-flow-rate bloodstream imaging mode.

3. The ultrasound diagnosis apparatus according to claim 1, wherein the processing circuitry is further configured to display, as text on a display screen, information that recommends the low-flow-rate bloodstream imaging mode when the second value of the flow rate indicates that the switch to the low-flow-rate bloodstream imaging mode from the bloodstream imaging mode should be performed.

4. The ultrasound diagnosis apparatus according to claim 1, wherein an imaging method differs between the bloodstream imaging mode and the low-flow-rate bloodstream imaging mode, and the imaging method is defined by a scan protocol and a signal processing method.

5. An ultrasound diagnosis apparatus, comprising processing circuitry configured to:
acquire a change in a display parameter related to image display in a first bloodstream imaging method during execution of the first bloodstream imaging method, wherein the ultrasound diagnosis apparatus includes a bloodstream imaging mode of displaying a bloodstream signal acquired by an ultrasonic probe, wherein the bloodstream imaging mode includes the first bloodstream imaging method and a second bloodstream imaging method differing from each other in at least one of a scan protocol or signal processing; and
determine whether or not the display parameter after the change satisfies a predetermined condition.

6. The ultrasound diagnosis apparatus according to claim 5, wherein the processing circuitry is further configured to transition from the first bloodstream imaging method to the second bloodstream imaging method when the display parameter after the change satisfies the predetermined condition.

7. The ultrasound diagnosis apparatus according to claim 6, wherein the processing circuitry is further configured to display information indicating that a transition is made from the first bloodstream imaging method to the second bloodstream imaging method.

8. The ultrasound diagnosis apparatus according to claim 5, wherein the processing circuitry is further configured to display information regarding whether or not to transition from the first bloodstream imaging method to the second bloodstream imaging method.

9. The ultrasound diagnosis apparatus according to claim 8, wherein the processing circuitry is further configured to transition from the first bloodstream imaging method to the second bloodstream imaging method when a transition command is issued.

10. The ultrasound diagnosis apparatus according to claim 7, wherein the processing circuitry is further configured to display information indicating that a transition has been made from the first bloodstream imaging method to the second bloodstream imaging method.

11. The ultrasound diagnosis apparatus according to claim 5, wherein the first bloodstream imaging method displays flow rate information of a bloodstream or power information of a bloodstream, and the second bloodstream imaging method displays flow rate information of a bloodstream or power information of a bloodstream.

12. The ultrasound diagnosis apparatus according to claim 1, wherein the processing circuitry is further configured to compare the second value of the display parameter with the threshold.

13. The ultrasound diagnosis apparatus according to claim 1, wherein the processing circuitry is further configured to:
receive a change in the threshold; and
compare the changed flow rate range with the changed threshold.

14. The ultrasound diagnosis apparatus according to claim 1, wherein the processing circuitry is further configured to:
acquire another changed flow rate range after the first transition during execution of the low-flow-rate bloodstream imaging method; and
cause the second transition from the low-flow-rate bloodstream imaging method to the first bloodstream imaging method when determining that the another changed flow rate range is greater than the threshold value.

15. The ultrasound diagnosis apparatus according to claim 2, wherein the processing circuitry is further configured to display the text at a position not overlapping with a region of interest (ROI) on an ultrasonic image.
